(19) United States
(12) Patent Application Publication
(10) Pub. No.: US 2026/0335334 A1
(43) Pub. Date: Oct. 29, 2026

(54) STABLE AQUEOUS MIDAZOLAM FORMULATION FOR INTRANASAL DELIVERY

(71) Applicant: Amphastar Pharmaceuticals, Inc., Rancho Cucamonga, CA (US)

(72) Inventors: Jack Yongfeng ZHANG, Rancho Cucamonga, CA (US); Ray Lameng LEI, Rancho Cucamonga, CA (US); Ying WANG, Rancho Cucamonga, CA (US); Mary Zi-ping LUO, Rancho Cucamonga, CA (US)

(21) Appl. No.: 19/189,558

(22) Filed: Apr. 25, 2025

(51) Int. Cl. A61K 31/5517; A61K 9/00; A61K 47/02; A61K 47/10; A61K 47/18; A61K 47/26

(52) U.S. Cl. CPC A61K 31/5517; A61K 9/0043; A61K 47/02; A61K 47/10; A61K 47/183; A61K 47/186; A61K 47/26

(57) ABSTRACT

The present disclosure describes an aqueous solution formulation for the delivery of a therapeutic benzodiazepine compound, midazolam, to the nasal mucosa, to be used as a sedative/anxiolytic prior to surgical or other medical procedures. The pharmaceutical composition of midazolam hydrochloride, at concentrations of 5 mg/mL to 40 mg/mL midazolam base, is in a matrix, which is mainly water (which is greater than or equal to 75% w/w), along with ethanol, propylene glycol, polysorbate 20 (polyoxyethylene (20) sorbitan monolaurate), benzalkonium chloride, EDTA, sodium chloride, and sufficient phosphate buffer agents to provide a solution with a pH at 3.2 to 4.2. The compositions have excellent physical and chemical stability.

### FIELD OF THE INVENTION

This disclosure relates to aqueous formulations for the delivery of medications suitable for intranasal administration. M ore particularly, the present disclosure pertains stable aqueous formulations containing midazolam hydrochloride in water along with ethanol, propylene glycol, polysorbate 20 (polyoxyethylene (20) sorbitan monolaurate), benzalkonium chloride, EDTA, sodium chloride, and sufficient phosphate buffer agents for use in an intranasal device as a sedative/anxiolytic prior to surgical or other medical procedures.

### BACKGROUND OF THE INVENTION

Midazolam is a benzodiazepine that belongs to a group of central nervous system depressants. It is known for its anesthetic, sedative, anxiolytic, anticonvulsant, and muscle relaxant properties for the treatment of anesthesia, anxiety disorders, and acute seizures. When compared to other benzodiazepines, midazolam has the unique characteristic of having a rapid onset of action and short duration of action based on the method of administration, making it optimal for quick treatments. Midazolam first came into medical use in the 1980s with the development of different delivery methods. While oral and buccal methods are available, they are not preferred due to factors such as the inactivation of the drug from first-pass metabolism and insufficient drug strength respectively (Lingamchetty, et al.). Asa result, midazolam is primarily administered via injectable or intranasal means with varying advantages and disadvantages.

Injection of midazolam (e.g., intravenous or intramuscular) provides a rapid and strong systemic effect; however, these options have several disadvantages that make them less ideal. Injectable delivery methods require hospitalization or the presence of skilled medical personnel for successful administration. Additionally, injections can be irritating and painful resulting in discomfort to the patient; particularly, pediatric patients display low compliance with injections.

In contrast to the injection strategies for midazolam, intranasal delivery is non-invasive and does not require special training for administration, reducing the risk of injury and improving patient comfort and compliance. Additional advantages of intranasal delivery result from the anatomy of the nasal cavity. The nasal cavity consists of highly vascularized epithelial tissue for multiple purposes including protection and absorption. The vascular nature of the nasal cavity is a product of the pervasive network of blood capillaries under the mucosal surface. The extensive blood vessel network gives the nasal cavity its characteristic of being a highly permeable site (Thorat, 2016) making the nasal mucosa an ideal site for rapid and strong systemic absorption of drugs such as midazolam.

Despite advantages over injectable midazolam, intranasal midazolam formulations have unique challenges that must be resolved to be effective. One such challenge is that the formulation must account for the small volume of the nasal cavity. Another challenge is the pH-dependent solubility of midazolam that must be addressed for an intranasal delivery system. Resolving these challenges requires using excipients. Consequently, another challenge becomes minimizing the irritation in patients associated with excipients for intranasal midazolam delivery.

The small size of the nasal cavity necessitates a low drug delivery volume of 150 to 200 μL (Pires, et al.) to avoid swallowing through postnasal drip and sneezing or coughing, which would expel a portion of the medication dose. Additionally, the solubility of midazolam in water is pH dependent. Under acidic conditions, midazolam is present as an equilibrium mixture of the closed-ring form and the open-ring form caused by the acid-catalyzed ring opening of the 4,5-double bond of the diazepine ring (see FIG. 1). The open-ring molecule is polar and has excellent solubility at a pH of around 3.0 but is pharmacologically inactive. In physiological conditions, e.g., in environments with a pH of 5 to 8, the ring closes. The midazolam molecule then becomes active and is readily lipid soluble, allowing it to rapidly pass through the blood-brain barrier (Prommer).

The percentage of midazolam present in the open-ring form changes as a function of pH in aqueous solutions. As indicated in FIG. 2, the amount of open-ring compound is sensitive to changes in pH over 2.5 to 3.7 for both 1 mg/mL and 5 mg/mL concentrations. Above pH 5, at least 99% of the mixture is present in the closed-ring form.

The low water solubility of midazolam in neutral pH presents a challenge in creating a formulation that has an effective drug strength in a small volume to achieve the desired effects. Moreover, the epithelial tissue secretes mucus that coats the nasal cavity surfaces. It acts as protection from foreign entities, including drugs such as midazolam, by entrapping and expelling them through ciliary movement. A formulation for intranasal midazolam that is biocompatible with mucus would maximize the bioavailability of the drug and deliver a dose of the drug that is sufficient to produce the desired therapeutic effect.

The existing strategy to circumvent the pH-dependent nature of midazolam's solubility is through the application of a solvent system created with excipients from an extensive list of co-solvents and solubilizers. The system requires solvents and solubilizers that are organic—namely, carbon-based—compounds. The organic excipients create an organic matrix in which the midazolam is dissolved. However, Deot, et al. (2019) report that organic solvents and hyper-osmolality cause local irritation, increasing the safety risk during chronic use. Furthermore, organic matrices are often more viscous than mucus, with a less stable atomization effect and less consistent droplet size distribution, which may not be able to reach a wider and more consistent drug distribution within the nasal cavity for local or systemic application (Deot, et al.).

Lui, et al. (1991) disclose a midazolam formulation containing 11.1 mg/mL midazolam hydrochloride solution in water with the organic compound methocel at 1.5% (w/v). The formulation has an approximate pH of about 4. The study compared the absorption rate of intranasal midazolam to oral midazolam in dogs and found that the intranasal route had a faster mean intranasal absorption rate and a higher mean dose-normalized peak concentration. However, the use of methocel makes the formulation viscous, potentially hindering the capacity for consistent drug distribution to a patient's nasal cavity as well as causing discomfort.

Loftsson, et al. (2001) utilized cyclodextrin in the formulation. Their formulation included 17 mg/mL midazolam in water with 14% (w/v) sulfobutylether-p-cyclodextrin sodium salt and other excipients at a pH of about 4.3. Similarly, Gudmundsdottier, et al. (2001) disclosed a midazolam formulation for intranasal administration that also required water and cyclodextrin. Both studies observed that using high concentrations of cyclodextrin can cause damage to the nasal mucosa and irritation.

Wermeling, et al. (2006) studied the effects of an intranasal midazolam on twelve subjects. The studied formulation contained 25 mg/mL midazolam in polyethylene glycol (PEG) 400 and propylene glycol, both of which are organic compounds, making an organic matrix. The subjects reported experiencing nasal irritation. Knoester, et al. also studied an intranasal formulation of 30.9 mg/mL midazolam, which comprised water and a high percentage of propylene glycol at 25% v/v as co-solvents. A high proportion of propylene glycol, however, was shown to possibly cause a strong ciliostatic effect, potentially adding to the irritation felt by patients (Merkus, et al., 2001). Wermeling, et al. concluded that new strategies addressing irritation are needed.

U.S. Pat. No. 7,700,588 assigned to Merkus discloses pharmaceutical compositions containing midazolam in high concentrations for intranasal delivery. The presented composition contains midazolam in an organic matrix of water, glycerol, povidone, and polyethylene glycol 400. The composition has a pH of about 3 to 4.

U.S. Pat. No. 9,687,495 assigned to Gizurarson, with the brand name “Nayzilam®”, discloses an organic composition of intranasal midazolam that also contains water, ethanol, propylene glycol, PEG-6 methyl ether (mPEG 350), polyethylene glycol 400 (PEG 400), and phenethyl alcohol. The formulation has a viscosity that ranges from about 1.5 cP to about 60 cP, with a pH range of about 4.5 to about 8.5.

The use of an organic matrix in past intranasal midazolam formulations enabled the delivery of effective amounts of the drug through the nasal cavity; however, the common problem of causing nasal irritation remains unaddressed. Pires, et al. (2022) report that organic solvents have been associated with irritation in the nasal cavity by patients, such as burning and general discomfort. Reducing organic solvents may resolve the nasal irritation associated with intranasal midazolam.

Aqueous formulations offer advantages for intranasal midazolam formulations over organic matrices. Aqueous formulations, ones that do not use an organic matrix and have a high percentage of water, are less viscous and show a more stable atomization effect in terms of consistent droplet size distribution, which is desirable for nasal delivery. Minimizing organic solvents also makes aqueous solvent formulations less irritating to the nasal cavity. Behl, et al. (1998), however, assert that the solubility of the therapeutic agent in water may limit the effectiveness of nasal administration. Without organic matrices as the primary solvent strategy, aqueous formulations must rely on the pH-dependent solubility characteristic of midazolam.

Acidifying agents and pH buffers as excipients aid in solubilizing midazolam by creating an acidic environment for midazolam to be present in its salt form and suitable for intranasal delivery. When selecting the best acids for this purpose, a significant feature that should be accounted for is the excipients' potential to cause damage or irritation to the nasal mucosa of the user.

U.S. Patent Application Publication No. 2021/0379080 discloses a formulation of intranasal midazolam that is largely devoid of organic solvents and instead relies on a solvent system that comprises water, methoxy-polyethylene glycol, polyethylene glycol, propylene glycol, and ethanol or a combination thereof with hydrochloric acid and lactic acid as acidifying agents.

Certain acids can potentially cause irritation when used in high concentrations. For example, hydrochloric acid is known to cause burning and scarring on mucus membranes. Lactic acid, similarly, has been observed to cause redness, swelling, burning, and itchiness in patients and is suggested by healthcare professionals to not be used in the mouth or nose. A careful selection of acids is necessary to address the primary concern of irritation for intranasal midazolam.

The challenge in developing an aqueous midazolam solution is delivering a therapeutically effective amount of midazolam appropriate for intranasal delivery while minimizing irritation in the user. Therefore, there is an unmet medical need for an intranasal midazolam formulation that is more biocompatible to mucus, thus minimizing irritation to the user. The instant disclosure presents therapeutically effective aqueous intranasal midazolam formulations that are stable and accomplish these goals by possessing comparable osmolality, viscosity, and density to saline.

### SUMMARY OF THE INVENTION

The present disclosure provides a formulation of a liquid solution suitable for intranasal delivery of a therapeutically effective amount of midazolam that can be used as a sedative or anxiolytic prior to surgical or other medical procedures. The disclosure is based on the discovery of an aqueous intranasal formulation of midazolam without a high concentration of organic co-solvents and solubilizers. The high water content of the aqueous midazolam formulation is more biocompatible with mucus, decreasing the likelihood of strong irritation compared to formulations containing high amounts of organic solvents.

The formulations are aqueous solutions that can be placed in an intranasal device and that contain midazolam hydrochloride in a solution of co-solvents, a surfactant, a preservative, a stabilizing agent, an isotonic agent, and pH buffer agents in water. In one embodiment, the preferred co-solvents are ethanol and propylene glycol. In another embodiment, the preferred surfactant is polysorbate 20 (polyoxyethylene (20) sorbitan monolaurate). In further embodiments, the preferred preservative is benzalkonium chloride. In alternate embodiments, the preferred stabilizing agent is ethylenediaminetetraacetic acid (EDT A). In additional embodiments, the preferred isotonic agent is sodium chloride. In certain embodiments, the preferred pH buffer agents are sodium phosphate monobasic and phosphoric acid.

As noted above, midazolam is not water-soluble in its closed-ring form, which is present in neutral pH environments but becomes readily water-soluble in its open-ring, salt form in acidic pH ranges around 3.0. The formulations of the disclosure leverage this relationship by using acidifying agents, which also act as pH regulators, to dissolve a high concentration of midazolam that is needed to be delivered in a small volume that is appropriate for the nasal cavity.

The formulations of the disclosure are stable aqueous formulations of intranasal midazolam. Over a storage period of 3 months at 4C, 25 °C, and 40 °C, the buffer system of the formulations of the disclosure demonstrated a stable pH. Furthermore, the disclosed aqueous intranasal midazolam formulations with the preferred buffer system are physically and chemically stable over a 3-month period at 4C, 25 °C, and 40 °C with total impurities not more than 0.30%.

The intranasal formulations of midazolam minimize nasal irritation and discomfort in the patient that can be associated with the use of certain pH buffer systems or the presence of organic excipients at a high percentage of the solution. It is acknowledged that special attention should be applied when determining the most favorable acidifying agents that contain the buffer system to avoid irritating effects on the nasal mucosa. Decreasing the proportion of organic excipients in the solution allows for the formulation to be higher in water, making it more biocompatible with the mucus in the nasal cavity and minimizing irritation.

In one embodiment, the aqueous pharmaceutical formulation of midazolam solution for intranasal administration contains: midazolam, or a pharmaceutically acceptable salt form; not less than 75% (w/w) of water relative to the total weight of the formulation; a co-solvent or a combination of co-solvents selected from the group consisting of ethanol, isopropyl alcohol, propylene glycol, ethylene glycol, glycerol, povidone, 2-(2-ethoxyethoxy)ethanol, or combinations thereof; a surfactant selected from the group consisting of polyethylene glycols, cyclodextrins, polysorbates, and sorbitan oleates; a preservative selected from the group consisting of phenol, benzyl alcohol, benzalkonium chloride, and vitamin E; a stabilizing agent selected from the group consisting of ethylenediaminetetraacetic acid (EDTA), ethylglycoltetraacetic acid (EGTA), citric acid or combinations thereof; an isotonic agent selected from the list consisting of sodium chloride, potassium chloride, calcium chloride, or a mixture thereof; and pH buffer agents selected from the group consisting of citrate buffer, acetate buffer, phosphate buffer, or mixtures thereof in an amount sufficient to adjust the solution pH to between about 3.2 to about 4.2. In certain embodiments, the midazolam, or a pharmaceutically acceptable salt form thereof, is midazolam hydrochloride, which can be present in a concentration of about 5 mg/mL to about 40 mg/mL.

In one embodiment, the co-solvent is a combination of ethanol and propylene glycol. In an embodiment, the combination of ethanol and propylene glycol is present in an amount of about 0.1% to about 20% (w/w) relative to the total weight of the formulation. In a further embodiment, the surfactant is polysorbate 20 (polyoxyethylene (20) sorbitan monolaurate). In an alternate embodiment, the polysorbate 20 (polyoxyethylene (20) sorbitan monolaurate) is present in an amount of about 0.1% to about 20% (w/w) relative to the total weight of the formulation. In a further embodiment, the preservative is benzalkonium chloride. In yet another embodiment, the benzalkonium chloride is present in an amount not more than about 0.01% (w/w) relative to the total weight of the formulation. In an embodiment, the stabilizing agent is ethylenediaminetetraacetic acid (EDTA). In another embodiment, the EDTA is present in an amount not more than about 0.02% (w/w) relative to the total weight of the formulation. In an alternate embodiment, the isotonic agent is sodium chloride. In yet another embodiment, the sodium chloride is present in an amount not more than about 0.9% (w/w) relative to the total weight of the formulation. In certain embodiments, the mixture of pH buffer agents includes sodium phosphate monobasic and phosphoric acid. In some embodiments, the sodium phosphate monobasic is present in an amount of about 0.01% to about 1.0% (w/w) relative to the total weight of the formulation, and phosphoric acid is present in an amount sufficient to adjust the formulation pH to be between 3.2 and 4.2. In one embodiment, the formulation is delivered by an intranasal device at a dose of about 0.1 mg to about 5 mg of midazolam or by an intranasal device at the volume of about 0.1 mL to about 0.7 mL.

In further embodiments, the density of the aqueous pharmaceutical formulation of midazolam solution is similar to that of water of about 1 g/mL and not more than about 1.05 g/mL. In other embodiments, the viscosity of the solution is between about 1 cP to about 10 cP. In yet another embodiment, the osmolality of the solution is between about 290 mOsm/kg to about 400 mOsm/kg.

Another embodiment of the disclosure is directed to an aqueous pharmaceutical formulation of midazolam solution for intranasal administration containing: midazolam hydrochloride in a solution at the concentration of midazolam between about 10 mg/mL to about 20 mg/mL; water in an amount ranging from about 75% to about 95% (w/w) relative to the total weight of the formulation; ethanol in an amount ranging from about 9% to about 10% (w/w) relative to the total weight of the formulation; propylene glycol in an amount ranging from about 7% to about 8% (w/w) relative to the total weight of the formulation; polysorbate 20 (polyoxyethylene (20) sorbitan monolaurate) in an amount not more than about 2.5% (w/w) relative to the total weight of the formulation; benzalkonium chloride in an amount ranging from about 0.001% to about 0.01% (w/w) relative to the total weight of the formulation; EDTA in an amount ranging from about 0.001% to about 0.02% (w/w) relative to the total weight of the formulation; sodium chloride in an amount ranging from about 0.1% to about 0.9% (w/w) relative to the total weight of the formulation; and sodium phosphate monobasic and phosphoric acid wherein sodium phosphate monobasic is in an amount ranging from about 0.1% to about 1.0% (w/w) relative to the total weight of the formulation, and wherein phosphoric acid in an amount sufficient to adjust the solution pH to be between about 3.2 and about 4.2.

In one embodiment, the aqueous pharmaceutical formulation of midazolam solution for intranasal administration contains: midazolam hydrochloride in a solution at the concentration of midazolam of about 10 mg/mL; water in an amount of about 78.8% (w/w) relative to the total weight of the formulation; ethanol in an amount of about 9.5% (w/w) relative to the total weight of the formulation; propylene glycol in an amount of about 7.3% (w/w) relative to the total weight of the formulation; polysorbate 20 (polyoxyethylene (20) sorbitan monolaurate) in an amount of about 2.39% (w/w) relative to the total weight of the formulation; benzalkonium chloride in an amount of about 0.01% (w/w) relative to the total weight of the formulation; EDTA in an amount of about 0.02% (w/w) relative to the total weight of the formulation; sodium chloride in an amount of about 0.51% (w/w) relative to the total weight of the formulation; and sodium phosphate monobasic and phosphoric acid wherein sodium phosphate monobasic in an amount of about 0.36% (w/w) relative to the total weight of the formulation and phosphoric acid in an amount sufficient to adjust the solution pH to be between about 3.5 to about 3.8. In another embodiment, the aqueous pharmaceutical formulation of midazolam solution for intranasal administration contains: midazolam hydrochloride in a solution at the concentration of midazolam of 20 mg/mL; water in an amount of about 77.7% (w/w) relative to the total weight of the formulation; ethanol in an amount of about 9.5% (w/w) relative to the total weight of the formulation; propylene glycol in an amount of about 7.3% (w/w) relative to the total weight of the formulation; polysorbate 20 (polyoxyethylene (20) sorbitan monolaurate) in an amount of about 2.39% (w/w) relative to the total weight of the formulation; benzalkonium chloride in an amount of about 0.01% (w/w) relative to the total weight of the formulation; EDTA in an amount of about 0.02% (w/w) relative to the total weight of the formulation; sodium chloride in an amount of about 0.51% (w/w) relative to the total weight of the formulation; and sodium phosphate monobasic and phosphoric acid wherein sodium phosphate monobasic in an amount of about 0.36% (w/w) relative to the total weight of the formulation and phosphoric acid in an amount sufficient to adjust the solution pH to be between about 3.5 to about 3.8.

In certain embodiments, the formulation is stable at 4C, 25 °C, and 40 °C with total impurities not more than 0.30% after 3 months of storage.

Other features and advantages of the invention will be apparent from the detailed description and examples that follow.

### BRIEF DESCRIPTION OF THE DRAWINGS

The foregoing background, as well as the following summary, detailed description, and examples of the invention, will be better understood when read in conjunction with the appended figures. The purpose of the figures is to provide a visual aid in the understanding of the theory of midazolam characteristic relationships. To illustrate the invention, the figures demonstrate embodiments of the present invention; however, it should be understood that the invention is not limited to the exact arrangements, examples, and instrumentalities provided.

FIG. 1 shows the equilibrium structures of the closed-ring and the open-ring forms of midazolam.

FIG. 2 is a chart depicting the relationship between the percentage of the open-ring form of midazolam present and a changing pH environment. Specifically, FIG. 2 shows the percentage of the open-ring form of midazolam as a function of pH.

FIG. 3 is a chart comparing the midazolam solubility of various solutions at pH 3.5 to 4.2 at room temperature.

FIG. 4 is a chart comparing the pH stability of three different formulations with different buffer systems at 40 °C at the beginning and at the end of 3 months.

FIG. 5 is a chart comparing the total impurities percentage of three different formulations with different buffer systems at 40 °C at the beginning and at the end of 3 months.

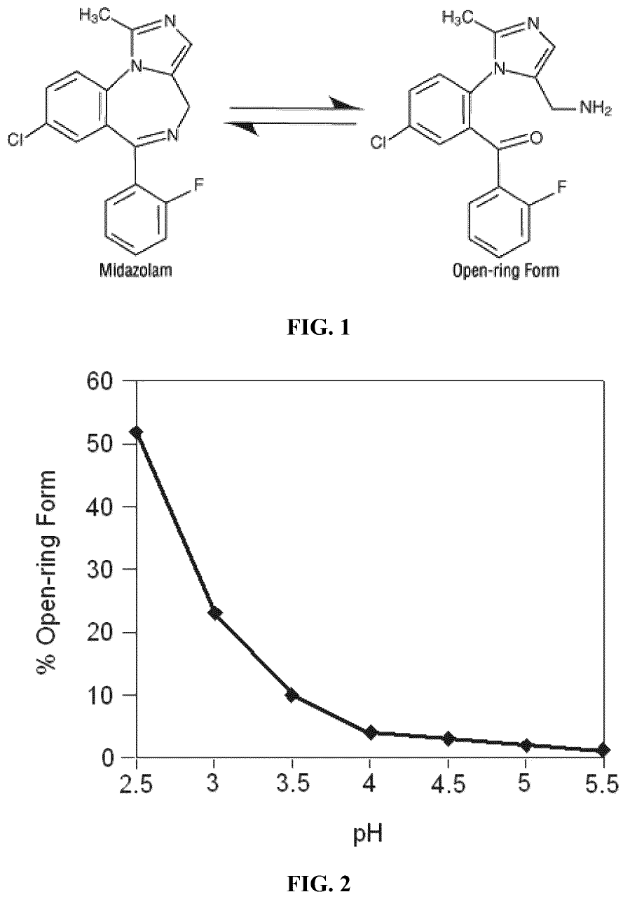

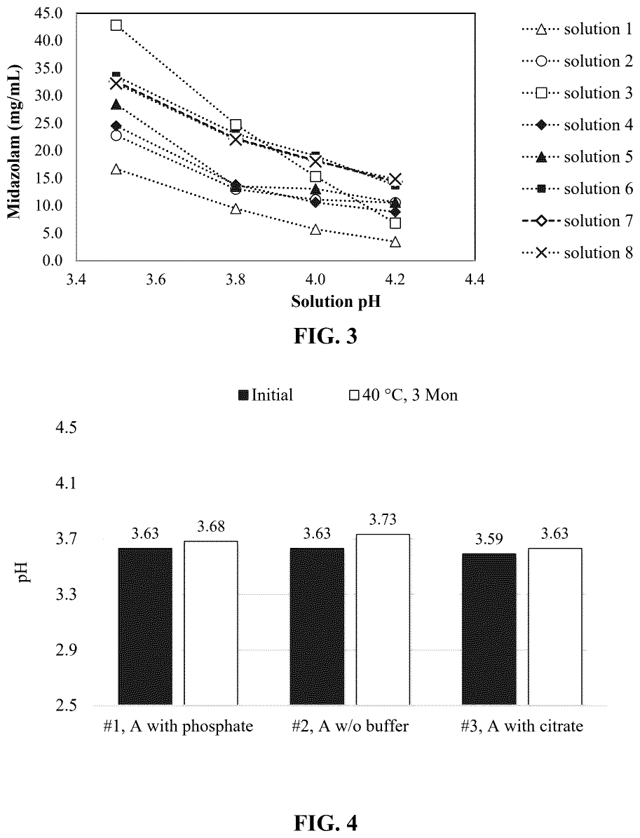

### DETAILED DESCRIPTION

The present disclosure is based on the discovery of aqueous solution formulations of midazolam with a water content of no less than 75% (w/w) relative to the total weight of the formulation that are suitable for intranasal delivery and have similar physical properties—particularly in water content, viscosity, and osmolality—to saline, indicating strong compatibility with mucus. As a result, the formulations will not cause strong irritation compared to midazolam intranasal formulations containing high amounts of organic excipients.

The formulations principally eliminate the reliance on organic matrices to dissolve a therapeutically effective amount of midazolam for intranasal delivery thereby decreasing the chances of nasal irritation associated with a high proportion of organic excipients. Rather, the disclosed intranasal midazolam formulations leverage the pH-dependent nature of midazolam's solubility. The favored pH-buffer systems of the disclosure focus on minimizing irritation in the nasal mucosa while also being stable in storage conditions. Furthermore, the disclosed intranasal midazolam formulations containing a low proportion of organic co-solvents and solubilizers and a high proportion of water do not compromise the effectiveness of delivering the intended dose of midazolam for therapeutic effect.

The reduction in the proportion of organic excipients has led to the surprising discovery of a formulation that has similar physical properties to saline while maintaining the therapeutic effectiveness of intranasal midazolam. Particularly, the formulations of the disclosure have comparable osmolality, viscosity, and density to saline which is biocompatible with the mucus present in the nasal cavity resulting in a lower risk of irritation compared to formulations that use a high proportion of organic solvents. The formulations based on this disclosure are stable aqueous intranasal solutions of midazolam free from high concentrations of organic co-solvents, reducing nasal irritation due to its high water content and similar physical properties to saline.

The general description and the following detailed description are exemplary and explanatory only and are not restrictive of the invention, as defined in the appended claims. Other aspects of the present invention will be apparent to those skilled in the art in view of the detailed description of the invention as provided herein.

For clarity of disclosure, and not by way of limitation, the detailed description of the invention is divided into subsections that describe or illustrate certain features, embodiments, or applications of the present invention.

### DEFINITIONS

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

It is also to be understood that the terminology used herein is for the purpose of describing particular embodiments only and is not intended to be limiting.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

Throughout this specification, unless the context requires otherwise, the word “comprise” or variations such as “comprises” or “comprising,” will be understood to imply the inclusion of a stated integer or group of integers but not the exclusion of any other integer or group of integers.

As used herein, the term “consisting of” excludes any element, step, or ingredient not specified in the claim element.

The term “aqueous” used herein is defined as water being present in a proportion that is no less than 75% w/w relative to the total weight of the formulation of the midazolam containing solution.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. W here the stated range includes one or both of the limits, ranges excluding either or both of those included limits are also included in the invention.

It is also to be understood that the terminology used herein is for the purpose of describing particular embodiments only and is not intended to be limiting.

A “subject” or “patient,” as used therein, may be a human or non-human mammal. Non-human mammals include, for example, livestock and pets, such as ovine, bovine, porcine, canine, feline and murine mammals. Preferably, the subject is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

### AQueous Solution Formulation of Midazolam

One aspect of the disclosure is directed to aqueous solutions containing midazolam that do not contain a high concentration of organic co-solvents and solubilizers. The aqueous formulations of the disclosure contain no less than 75% w/w of water relative to the total weight of the formulation. Due to the high water content, the aqueous formulations are more biocompatible with mucus, decreasing the likelihood of strong irritation compared to formulations containing high amounts of organic solvents.

The formulations generally contain midazolam, or a pharmaceutically acceptable salt form, such as midazolam hydrochloride, not less than 75% (w/w) of water relative of the total weight of the formulation; one or more co-solvent, a surfactant, a preservative, a stabilizing agent, an isotonic agent, and a pH buffer agent. In certain embodiments, the formulations are aqueous solutions in an intranasal device and essentially contain midazolam hydrochloride in a solution of co-solvents, a surfactant, a preservative, a stabilizing agent, an isotonic agent, and pH buffer agents in water.

The formulations according to the disclosure are preferably suitable for intranasal administration. In certain embodiments, the formulations are in an intranasal device.

The active pharmaceutical ingredient in the formulation is midazolam base or midazolam hydrochloride. The concentration of midazolam in the formulations is from about 5 mg/mL to about 40 mg/mL, alternatively from about 10 mg/ml to about 20 mg/ml, alternatively from about 10 mg/mL to about 40 mg/mL. Preferably, the concentration of midazolam in the formulations is from 10 mg/mL to 20 mg/mL. M ore preferably, the concentrations of midazolam in the formulations are about 10 mg/mL and about 20 mg/mL.

The formulations contain at least about 75% (w/w) or not less than about 75% (w/w) of water relative to the total weight of the formulation. Without being bound by theory, the water can act as the solvent. In certain embodiments, the formulations contain from about 75% to about 95% (w/w), alternatively from about 75% to about 85% (w/w), alternatively from about 75% to about 80% (w/w), alternatively from about 77% to about 90% (w/w), alternatively from about 77% to about 85% (w/w), from about 77% to about 82% (w/w) of water relative to the total weight of the formulation.

Midazolam solubility in aqueous solutions of the present disclosure is facilitated by the addition of a co-solvent and a surfactant. The co-solvents utilized in the formulation can be selected from ethanol (EtOH), isopropyl alcohol, propylene glycol, ethylene glycol, glycerol, povidone, 2-(2-ethoxyethoxy)ethanol, or combinations thereof. In an embodiment, the formulations contain ethanol, glycerol, and/or propylene glycol as the co-solvent. In some embodiments, the only co-solvents in the formulations are ethanol and propylene glycol. In another embodiment, the only co-solvents in the formulations are ethanol and glycerol. In further embodiments, the formulations contain from about 3% to about 20% (w/w), alternatively from about 5% to about 20% w/w, alternatively from about 10% to about 20% (w/w) of co-solvent relative to the total weight of the formulation.

In certain embodiments, the co-solvent is ethanol present in an amount from about 0.1% to about 20% (w/w) relative to the total weight of the formulation. In certain embodiments, the co-solvent is propylene glycol present in an amount from about 0.1% to about 20% (w/w) relative to the total weight of the formulation. In certain embodiments, the co-solvent is a combination of ethanol and propylene glycol from about 0.1% to about 20% (w/w) relative to the total weight of the formulation. Preferably, the co-solvent is a combination of ethanol present in an amount from about 3.4% to about 20% (w/w) relative to the total weight of the formulation and propylene glycol present in an amount from about 5% to about 20% (w/w) relative to the total weight of the formulation. M ore preferably, the co-solvent is a combination of ethanol present in an amount from about 9% to about 10% (w/w) relative to the total weight of the formulation and propylene glycol present in an amount from about 7% to about 8% (w/w) relative to the total weight of the formulation.

A surfactant is added to improve the solution's stability and enhance the solubility of midazolam or midazolam salt. The surfactant utilized in the formulations can be selected from polyethylene glycols, cyclodextrins, polysorbates, and sorbitan oleates (such as, e.g., sorbitan mono-oleate and sorbitan trioleate), or a combination thereof. In some embodiments, the surfactant is selected from polysorbates, polyethylene glycols, cyclodextrin, or combinations thereof. Examples of suitable polysorbates include polysorbate 20 (polyoxyethylene (20) sorbitan monolaurate) and polysorbate 80 (polyoxyethylene (20) sorbitan monooleate). Examples of suitable polyethylene glycols include but are not limited to PEG 400, i.e., polyethylene glycol having an average molecular weight of 400, and PEG 3350, i.e. polyethylene glycol having an average molecular weight of 3350. Examples of suitable cyclodextrins include but are not limited to betadex sulfobutyl ether sodium (SBE-β-CD). The formulation can contain from about 2% to about 20% (w/w), alternatively from about 2% to about 15% (w/w), alternatively from about 2% to about 10% (w/w), alternatively from about 2% to about 7% (w/w), alternatively from about 2% to about 5% (w/w) of surfactant(s) relative to the total weight of the formulation.

In some embodiments, the formulations contain polysorbate 20 (polyoxyethylene (20) sorbitan monolaurate) as the only surfactant. In other embodiments, the formulations contain polysorbate 20 (polyoxyethylene (20) sorbitan monolaurate) and PEG 400 as the only surfactants. In further embodiments, the formulations contain polysorbate 80 (polyoxyethylene (20) sorbitan monooleate) as the only surfactant. In yet further embodiments, the formulations contain polysorbate 80 (polyoxyethylene (20) sorbitan monooleate) and PEG 400 as the only surfactants. In alternate embodiments, the formulations contain polysorbate 20 (polyoxyethylene (20) sorbitan monolaurate), PEG 400, and betadex sulfobutyl ether sodium as the only surfactant. In yet another embodiment, the formulations contain betadex sulfobutyl ether sodium as the only surfactant. In a further embodiment, the formulations contain betadex sulfobutyl ether sodium and polysorbate 80 (polyoxyethylene (20) sorbitan monooleate) as the only surfactants.

In certain embodiments, the surfactant is polysorbate 20 (polyoxyethylene (20) sorbitan monolaurate) (T20) present in an amount from about 0.1% to about 20% (w/w) relative to the total weight of the formulation. Preferably, the surfactant polysorbate 20 (polyoxyethylene (20) sorbitan monolaurate) is present in an amount not more than about 2.5% (w/w) relative to the total weight of the formulation. M ore preferably, the surfactant polysorbate 20 (polyoxyethylene (20) sorbitan monolaurate) is about 2.39% (w/w) relative to the total weight of the formulation.

A preservative and a stabilizing agent are added to control the aqueous formulation's chemical stability and to prevent bacterial contamination. The preservative utilized in the formulations can be selected from phenol, benzyl alcohol, benzalkonium chloride, vitamin E, and a combination thereof. In certain embodiments, the formulations contain benzyl alcohol and/or benzalkonium chloride as the preservative. In further embodiments, the formulations contain benzyl alcohol as the preservative or as the only preservative. In additional embodiments, the formulations contain benzalkonium chloride as the preservative or as the only preservative.

In certain embodiments, the preservative is benzalkonium chloride in an amount that is not more than about 0.01% (w/w) relative to the total weight of the formulation. Preferably, benzalkonium chloride is present in an amount from about 0.001% to about 0.01% (w/w) relative to the total weight of the formulation. M ore preferably, benzalkonium chloride is about 0.01% (w/w) relative to the total weight of the formulation.

The stabilizing agent utilized in the formulations can be selected from ethylenediaminetetraacetic acid (EDTA), ethylglycoltetraacetic acid (EGTA), citric acid or combinations thereof. In certain embodiments, the formulations contain EDTA as the only stabilizer (stabilizing agent). The formulation can contain from about 0.001% to about 0.02% (w/w), alternatively from about 0.002 to about 0.02% (w/w), alternatively from about 0.001% to about 0.005% (w/w), alternatively from about 0.01% to about 0.004% (w/w), alternatively from about 0.01% to about 0.003% (w/w) of the stabilizing agent.

In certain embodiments, the stabilizing agent in the formulations is ethylenediaminetetraacetic acid (EDTA) in an amount not more than about 0.02% (w/w) relative to the total weight of the formulation. Preferably, EDTA is present in an amount from about 0.001% to about 0.02% (w/w) relative to the total weight of the formulation. M ore preferably, EDTA is about 0.02% (w/w) relative to the total weight of the formulation.

An isotonic agent, selected from a list comprising sodium chloride, potassium chloride, calcium chloride, and a mixture thereof, is added to the formulations. Without being bound by theory the isotonic agent may be necessary to adjust the osmolality of the solution. In certain embodiments, the isotonic agent is sodium chloride. In certain embodiments, the isotonic agent in the formulations is sodium chloride in an amount that is not more than about 0.9% (w/w) relative to the total weight of the formulation. Preferably, sodium chloride is present in an amount from about 0.01% to about 0.9% (w/w) relative to the total weight of the formulation. M ore preferably, sodium chloride is about 0.51% (w/w) relative to the total weight of the formulation.

To optimize the midazolam solubility in the aqueous formulations, the pH of the solution is controlled to be between about 3.2 and about 4.2 by adding buffer agents chosen from a list comprising citrate buffer, acetate buffer, phosphate buffer, or mixtures thereof. The disclosed list of buffer agents for the formulation is suitable for intranasal delivery of midazolam as they are not known to have a high potential for irritation. In certain embodiments, the buffer in formulations is a phosphate buffer, such as e.g., sodium phosphate monobasic.

In certain embodiments, the preferred buffer agent in the formulations is sodium phosphate monobasic in an amount from about 0.01% to about 1.0% (w/w) relative to the total weight of the formulation. Preferably, sodium phosphate monobasic is present in an amount from about 0.1% to about 1.0% (w/w) relative to the total weight of the formulation. M ore preferably, sodium phosphate monobasic is about 0.36% (w/w) relative to the total weight of the formulation. Phosphoric acid was used in an amount sufficient to adjust the pH of the solution to be between about 3.2 and about 4.2, alternatively between about 3.4 to about 3.8. Furthermore, the preferred buffer phosphate demonstrates notable pH stability over alternative buffer systems.

The density of the aqueous midazolam formulation for intranasal delivery was developed to minimize irritation in the nasal cavity of the patient. Denser solutions can impair the movement of cilia, decreasing the effectiveness of the cilia to move effectively for the mucociliary clearance process. To avoid discomfort during intranasal administration, the solution's density is comparable to that of water. In certain embodiments, the density of the solution is from about 1.0 g/mL and not more than about 1.05 g/mL.

The viscosity of the aqueous midazolam formulation for intranasal delivery was developed to reduce irritation in the nasal cavity of the patient. Similar to solutions with high density, solutions with high viscosity cause greater resistance which can impair the movement of the cilia. Solutions with high viscosity are less able to flow easily through the nasal passages leading to a sense of congestion in the user. In some embodiments, the viscosity of the solution is between about 1 cP to about 10 cP.

The osmolality of an intranasal solution should be relatively isotonic with mucus, which is around about 300 mOsm/kg, to avoid nasal discomfort during administration. In some embodiments, the osmolality of the solution is from about 290 mOsm/kg to about 400 mOsm/kg.

The delivered dose of the aqueous midazolam formulation for intranasal delivery is in an amount sufficient to provide a therapeutic effect as a sedative or anxiolytic before medical procedures. In some embodiments, the solution is delivered by an intranasal device at the dose of about 0.1 mg to about 5 mg of midazolam.

The small, limited size of the nasal cavity requires the drug delivery volume of the aqueous midazolam formulation to be low. A high drug delivery volume would result in portions of the solution being swallowed or expelled via coughing or sneezing; therefore, an efficient intranasal drug delivery system must be reasonably low. In some embodiments, the volume of the solution is from about 0.1 mL to about 0.7 mL.

In one embodiment, the aqueous pharmaceutical formulation of midazolam solution for intranasal administration contains: (a) midazolam, or a pharmaceutically acceptable salt form, such as e.g., midazolam hydrochloride; (b) not less than 75% (w/w) of water relative to the total weight of the formulation; (c) co-solvent or a combination of co-solvents selected from the group consisting of ethanol, isopropyl alcohol, propylene glycol, ethylene glycol, glycerol, povidone, 2-(2-ethoxyethoxy)ethanol, or combinations thereof; (d) a surfactant selected from the group consisting of polyethylene glycols, cyclodextrins, polysorbates, and sorbitan oleates, such as e.g., sorbitan mono-oleate and sorbitan trioleate; (e) a preservative selected from the group consisting of phenol, benzyl alcohol, benzalkonium chloride, and vitamin E; (f) a stabilizing agent selected from the group consisting of ethylenediaminetetraacetic acid (EDTA), ethylglycoltetraacetic acid (EGTA), citric acid, or combinations thereof; (g) an isotonic agent selected from the list consisting of sodium chloride, potassium chloride, calcium chloride, or mixtures thereof; and pH buffer agents selected from the list consisting of citrate buffer, acetate buffer, phosphate buffer, or mixtures thereof in an amount sufficient to adjust the solution pH to be between about 3.2 and about 4.2. In certain embodiments of the formulation, the midazolam hydrochloride is present in a concentration of about 5 mg/mL to about 40 mg/mL.

In embodiments, the co-solvent can be a combination of ethanol and propylene glycol, which can be present in an amount of about 0.1% to about 20% (w/w) relative to the total weight of the formulation. The surfactant can be polysorbate 20 (polyoxyethylene (20) sorbitan monolaurate), which can be present in an amount of about 0.1% to about 20% (w/w) relative to the total weight of the formulation. The preservative can be benzalkonium chloride, which can be present in an amount not more than about 0.01% (w/w) relative to the total weight of the formulation. The stabilizing agent can be ethylenediaminetetraacetic acid (EDTA), which can be present in an amount not more than about 0.02% (w/w) relative to the total weight of the formulation.

In certain embodiments, the isotonic agent is sodium chloride, which can be present in an amount not more than about 0.9% (w/w) relative to the total weight of the formulation. In other embodiments, the mixture of pH buffer agents includes sodium phosphate monobasic and phosphoric acid; the sodium phosphate monobasic can be present in an amount of about 0.01% to about 1.0% (w/w) relative to the total weight of the formulation and phosphoric acid can be present in an amount sufficient to adjust the formulation pH to be between about 3.2 and about 4.2. In some embodiments, the density of the solution is similar to that of water of 1 g/mL and not more than 1.05 g/mL. In further embodiments, the aqueous pharmaceutical formulation of midazolam solution, wherein the viscosity of the solution is between about 1 cP to about 10 cP. In other embodiments, wherein the osmolality of the solution is between about 290 mOsm/kg to about 400 mOsm/kg. In certain embodiments, the aqueous pharmaceutical formulation of midazolam solution is delivered by an intranasal device at the dose of about 0.1 mg to about 5 mg of midazolam. In other embodiments, the aqueous pharmaceutical formulation of midazolam solution is delivered by an intranasal device at a volume of about 0.1 mL to about 0.7 mL.

In one embodiment, the preferred aqueous pharmaceutical formulation of midazolam solution contains midazolam hydrochloride at a concentration between about 10 mg/mL to about 20 mg/mL, water in an amount ranging from about 75% to about 95% (w/w) relative to the total weight of the formulation, ethanol in an amount ranging from about 9% to about 10% (w/w) relative to the total weight of the formulation, propylene glycol in an amount ranging from about 7% to about 8% (w/w) relative to the total weight of the formulation, polysorbate 20 (polyoxyethylene (20) sorbitan monolaurate) in an amount not more than about 2.5% (w/w) relative to the total weight of the formulation, benzalkonium chloride in an amount ranging from about 0.001% to about 0.01% (w/w) relative to the total weight of the formulation, EDTA in an amount ranging from about 0.001% to about 0.02% (w/w) relative to the total weight of the formulation, sodium chloride in an amount ranging from about 0.1% to about 0.9% (w/w) relative to the total weight of the formulation, and sodium phosphate monobasic and phosphoric acid where sodium phosphate monobasic is in an amount ranging from about 0.1% to about 1.0% (w/w) relative to the total weight of the formulation and phosphoric acid is in an amount to adjust the solution pH to be between about 3.2 and about 4.2.

M ore preferably, in one embodiment, the aqueous pharmaceutical formulation of midazolam solution contains midazolam hydrochloride at a concentration of about 10 mg/mL or about 20 mg/mL, water in an amount of about 78.8% (w/w) or about 77.7% (w/w) relative to the total weight of the formulation, ethanol in an amount of about 9.5% (w/w) relative to the total weight of the formulation, propylene glycol in an amount of about 7.3% (w/w) relative to the total weight of the formulation, polysorbate 20 (polyoxyethylene (20) sorbitan monolaurate) in an amount of about 2.39% (w/w) relative to the total weight of the formulation, benzalkonium chloride in an amount of about 0.01% (w/w) relative to the total weight of the formulation, EDTA in an amount of about 0.02% (w/w) relative to the total weight of the formulation, sodium chloride in an amount of about 0.51% (w/w) relative to the total weight of the formulation, and sodium phosphate monobasic and phosphoric acid where sodium phosphate monobasic is in an amount of about 0.36% (w/w) relative to the total weight of the formulation and phosphoric acid is in an amount to adjust the solution pH to be between about 3.5 and about 3.8.

The stability of the formulations of the disclosure is appropriate for storage and transport. The formulation was studied at 4 °C, 25 °C, and 40 °C after 3 months of storage. A n impurity profile of the formulation was created to analyze the amount of impurities as a method of determining stability which is explored in later parts of the disclosure. In some embodiments, the total percentage of impurities of the formulation was not more than 0.30% after 3 months of storage at 4 °C, room temperature, and 40 °C.

The formulations of the disclosure when nasally administered can be used as a sedative or anxiolytic. In other embodiments, disclosure is directed to nasally administering the formulations of treat anxiety disorders and acute seizures.

Another aspect of the disclosure is directed to a nasal delivery device containing the formulations of the disclosure. In one embodiment, the delivery device includes a container holding the formulations of the disclosure.

In addition, the disclosure includes methods of preparing the formulations. The methods generally include: mixing water with the co-solvent, surfactant, preservative, stabilizing agent (stabilizer), and isotonic agent; adding the midazolam, or a pharmaceutically acceptable salt form, such as midazolam hydrochloride; and adjusting the pH of the solution to about 3.2 to about 4.2.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### EXAMPLES

### Example 1: Midazolam Solubility in Saline Solutions with Different Co-Solvents and Surfactants

Midazolam solubility in 80% (w/w) saline solutions was studied with various co-solvents and surfactants between pH 3.5 and 4.2. The data are listed in Table 1-1 (below) and FIG. 3.

Single Co-solvent or surfactant formulations. Solutions 1 to 4 contained a single co-solvent or surfactant each of PEG 400, T20, EtOH, or propylene glycol respectively. At pH 3.5, PEG 400, T20, EtOH, and propylene glycol can maximize the midazolam solubility to 16.7 mg/mL, 22.8 mg/mL, 42.8 mg/mL, and 24.5 mg/mL, respectively. The primary concern that arises with the use of a single co-solvent/surfactant, however, is midazolam solubility sharply decreases as the pH increases. While some solutions, namely Solution 3 with EtOH, displayed a high midazolam concentration at pH 3.5, the midazolam solubility dropped greatly with small increases in pH making it ill-suited and risky for practical use.

Formulations containing multiple co-solvents/surfactants. Solutions with multiple co-solvents/surfactants were studied as Solutions 5 to 8. The data in Table 1-1 and FIG. 3 establishes that solutions with multiple co-solvents and surfactants (Solutions 5 to 8) have less variation in midazolam concentrations than solutions with a single co-solvent or surfactant (Solutions 1 to 4) from pH 3.5 to pH 4.2. The lower variability in midazolam concentration of Solutions 5 to 8 indicates greater solubility stability without potential precipitation. The experimental data indicated that the combination of T20, EtOH, and propylene glycol in water at pH 3.6±0.1 would be suitable for the midazolam aqueous formulation.

TABLE 1-1
Midazolam Saturation Solubility in Various Solutions
at Room Temperature between pH 3.5 and 4.2

| | Solution Excipients, w/w % | | | | | Midazolam Concentration (mg/mL) at | | | |
|---|---|---|---|---|---|---|---|---|---|
| Solution | | | | Propylene | | Solution pH, at Room Temperature | | | |
| # | PEG 400 | T20 | EtOH | Glycol | Saline | 3.5 | 3.8 | 4.0 | 4.2 |
| 1 | 20 | x | x | x | 80 | 16.7 | 9.5 | 5.7 | 3.4 |
| 2 | x | 20 | x | x | 80 | 22.8 | 13.0 | 11.1 | 10.5 |
| 3 | x | x | 20 | x | 80 | 42.8 | 24.7 | 15.3 | 6.8 |
| 4 | x | x | x | 20 | 80 | 24.5 | 13.8 | 10.6 | 8.8 |
| 5 | x | 10 | 10 | x | 80 | 28.4 | 13.5 | 13.0 | 10.6 |
| 6 | x | 2.5 | 10 | 7.5 | 80 | 33.6 | 23.0 | 19.0 | 13.7 |
| 7 | x | 2.5 | 7.5 | 10 | 80 | 32.6 | 22.2 | 18.2 | 14.3 |
| 8 | x | 2.5 | 5.5 | 12 | 80 | 32.2 | 22.0 | 18.0 | 14.8 |

PEG 400: polyethylene glycol (PEG) 400
T20: Polysorbate 20 (polyoxyethylene (20) sorbitan monolaurate)
EtOH: ethanol

### Example 2: Aqueous Formulations of Midazolam for Intranasal Delivery

Twenty-one different aqueous formulations of midazolam for intranasal delivery were generated. The compositions of these formulations are listed in Table 2-1 (Formulation 1-10) and Table 2-2 (Formulation 11-21).

TABLE 2-1
Aqueous Formulations of Midazolam for Intranasal Delivery

| Formulation | | Components | 1 | 2 | 3 | 4 | 5 | 6 | 7 | 8 | 9 | 10 |
|---|---|---|---|---|---|---|---|---|---|---|---|---|
| API | | Midazolam | 21.0 | 22.0 | 22.0 | 21.0 | 22.0 | 22.0 | 22.0 | 22.0 | 22.0 | 21.0 |
| Conc. (mg/mL) | | HCl | | | | | | | | | | |
| Excipients | Surfactant | Polysorbate 20 | 5 | 6.7 | 3.3 | 0.8 | 5 | 10 | 6 | | 5 | |
| (% | | Polysorbate 80 | | | | | | | | 10 | | 5 |
| w/w) | | PEG 400 | 5 | 8.3 | 3.3 | 0.8 | 5 | 5 | 2 | | 5 | 5 |
| | | PEG 3350 | | | | | | | | | | |
| | | SBE-β-CD | | | | | | | | | 5 | |
| | Co-solvent | Propylene | | | | | 5 | | | | | |
| | | Glycol | | | | | | | | | | |
| | | Ethanol | | | 3.4 | 3.4 | 5 | 5 | 8 | 10 | 5 | 10 |
| | | Glycerol | | | | | | | 4 | | | |
| | | Transcutol | | | | | | | | | | |
| | Preservative | Benzyl Alcohol | 1 | | | | | | | | | |
| | | Benzalkonium | | | | | | | | 0.01 | 0.01 | 0.01 |
| | | chloride | | | | | | | | | | |
| | Stabilizer | EDTA | 0.02 | | | | | | | 0.002 | 0.002 | 0.002 |
| | pH buffer | Citric Acid | | | | | | | | | | |
| | | Sodium Citrate | | | | | | | | | | |
| | | 2H₂O | | | | | | | | | | |
| | | Acetic Acid | | | | | | | | | | |
| | | Sodium Acetate | | | | | | | | | | |
| | Tonicity | NaCl | | | 0.8 | | | | | | | |
| | agent | | | | | | | | | | | |

| | | | | | | | | | | | |
|---|---|---|---|---|---|---|---|---|---|---|---|
| Solvent | Water. % w/w | 88.98 | 85.00 | 89.20 | 95.00 | 80.00 | 80.00 | 80.00 | 79.99 | 79.99 | 79.99 |

| | | | | | | | | | | |
|---|---|---|---|---|---|---|---|---|---|---|
| Solution pH | 3.5 | 3.5 | 3.6 | 3.2 | 3.5 | 3.4 | 3.5 | 3.4 | 3.8 | 3.6 |

PEG 3350: polyethylene glycol (PEG) 3350
SBE-β-CD: Betadex Sulfobutyl Ether Sodium
Transcutol: 2-(2-Ethoxyethoxy) ethanol
EDTA: Ethylenediaminetetraacetic acid disodium salt dihydrate

TABLE 2-2
Aqueous Formulations of Midazolam for Intranasal Delivery

| Formulation | | Components | 11 | 12 | 13 | 14 | 15 | 16 | 17 | 18 | 19 | 20 | 21 |
|---|---|---|---|---|---|---|---|---|---|---|---|---|---|
| API | | Midazolam | 21.0 | 22.0 | 22.8 | 42.8 | 28.4 | 27.1 | 24.5 | 28.5 | 22.0 | 20.0 | 20.0 |
| Conc. (mg/mL) | | HCl | | | | | | | | | | | |
| Excipients | Surfactant | Polysorbate 20 | | | 20 | | 10 | 6.6 | | 2.5 | | | 2.39 |
| (% | | Polysorbate 80 | | 5 | | | | | | | 5 | | |
| w/w) | | PEG 400 | | | | | | 6.6 | | | | | |
| | | PEG 3350 | | | | | | | | | | 20 | |
| | | SBE-β-CD | 10 | 5 | | | | | | | | | |
| | Co-solvent | Propylene | | | | | | | 20 | 7.5 | 5 | | 7.3 |
| | | Glycol | | | | | | | | | | | |
| | | Ethanol | 10 | 10 | | 20 | 10 | 6.6 | | 10 | 5 | | 9.5 |
| | | Glycerol | | | | | | | | | | | |
| | | Transcutol | | | | | | | | | 5 | | |
| | Preservative | Benzyl Alcohol | | | | | | | | | | | |
| | | Benzalkonium | 0.01 | 0.01 | 0.01 | 0.01 | 0.01 | 0.01 | 0.01 | 0.01 | | | |
| | | chloride | | | | | | | | | | | |
| | Stabilizer | EDTA | 0.002 | 0.002 | 0.002 | 0.002 | 0.002 | 0.002 | 0.002 | 0.02 | | | |
| | pH buffer | Citric Acid | | | | | | | | 0.85 | | | |
| | | Sodium Citrate | | | | | | | | 0.46 | | | |
| | | 2H₂O | | | | | | | | | | | |
| | | Acetic Acid | | | | | | | | | | | 0.05 |
| | | Sodium Acetate | | | | | | | | | | | 0.01 |
| | Tonicity | NaCl | | | 0.7 | 0.7 | | 0.7 | | 0.36 | | | 0.6 |
| | agent | | | | | | | | | | | | |
| Solvent | Water. % w/w | | 79.99 | 79.99 | 79.29 | 79.29 | 79.99 | 79.49 | 79.99 | 78.30 | 80.00 | 80.00 | 80.15 |
| Solution pH | | | 4.1 | 3.8 | 3.5 | 3.5 | 3.5 | 3.5 | 3.5 | 3.6 | 3.5 | 3.1 | 3.6 |

SBE-β-CD: Betadex Sulfobutyl Ether Sodium
Transcutol: 2-(2-Ethoxyethoxy) ethanol
EDTA: Ethylenediaminetetraacetic acid disodium salt dihydrate

Formulations 1 to 21 were prepared by:
i. Mixing water, as the solvent solution, with the excipients at room temperature,
ii. Adding the corresponding amount of midazolam hydrochloride to the solution, and
iii. Adjusting the Solution pH with either HCl/NaOH or buffer agents.

The testing in this Example demonstrated that formulations containing 20 mg/mL to 40 mg/mL of midazolam suitable for intranasal administration can be prepared in a solution with a high proportion of water (greater than 75% w/w) at a pH of 3 to 4 with the addition of a small proportion of various surfactants and co-solvents. A preservative, stabilizing agent, and pH buffer agents were added to the formulation to keep the solution stable. An isotonic agent was added to adjust the solution's osmolality to enhance the performance of the nasal spray.

### Example 3: Aqueous Intranasal Midazolam Formulation with Phosphate Buffer

Formulation A (see Table 3-1 below) was prepared by mixing the solvent solution of ethanol, propylene glycol, polysorbate 20 (polyoxyethylene (20) sorbitan monolaurate), benzalkonium chloride, EDTA, sodium phosphate monobasic, sodium chloride, and water for 30 minutes at room temperature. The corresponding amount of midazolam hydrochloride was then added to the solution, and the pH was adjusted to be 3.6±0.1 with phosphoric acid to prepare a 10 mg/mL or 20 mg/mL midazolam aqueous solution, respectively.

TABLE 3-1
Formulation A

| | | 10 mg/mL | 20 mg/mL |
|---|---|---|---|
| | | Midazolam Solution | Midazolam Solution |
| Component | Function | Percent (% w/w) | Percent (% w/w) |
| Midazolam HCl | API | 1.112 | 2.224 |
| Water | Solvent | 78.79 | 77.68 |
| Ethanol | Co-solvent | 9.50 | 9.50 |
| Propylene Glycol | Co-solvent | 7.30 | 7.30 |
| Polysorbate 20 | Surfactant | 2.39 | 2.39 |
| Benzalkonium chloride | Preservative | 0.01 | 0.01 |
| EDTA | Stabilizing | 0.02 | 0.02 |
| | Agent | | |
| NaH₂PO₄ | pH Buffer | 0.36 | 0.36 |
| Phosphoric Acid, 85% | pH Buffer | Adjust pH to 3.6 | Adjust pH to 3.6 |
| Sodium Chloride | Isotonic | 0.51 | 0.51 |
| | Agent | | |

| | | |
|---|---|---|
| Osmolality (mOsm/kg) | 298 | 360 |

| | |
|---|---|
| pH | 3.6 ± 0.1 |
| Viscosity at 25 °C | 2.6 ± 0.1 cP |
| Solution Density | 1.008 ± 0.003 g/mL |
| Surface Tension, (dynes/cm) | 35.5 ± 0.1 |
| Electro Conductivity at 23 °C | 4.3 ± 0.1 ms/cm |

The osmolality of the Formulation Asolution with 10 mg/mL midazolam was calculated to be 298 mOsm/kg, and the osmolality of the Formulation Asolution with 20 mg/mL midazolam was calculated to be 360 mOsm/kg. The solution was evaluated for its density, viscosity, surface tension, and electro-conductivity, which are the major factors for intranasal solution. The density of Formulation A (both 10 mg/mL and 20 mg/mL) is 1.008±0.003 g/mL at room temperature and the viscosity is 2.6±0.1 cP at 25 °C. The surface tension of Formulation A (both 10 mg/mL and 20 mg/mL) is 35.5±0.1 dynes/cm at room temperature and the electro-conductivity is 4.3±0.1 ms/cm at 23 °C.

### Example 4: Intranasal Midazolam Formulation Stability with Different Buffers

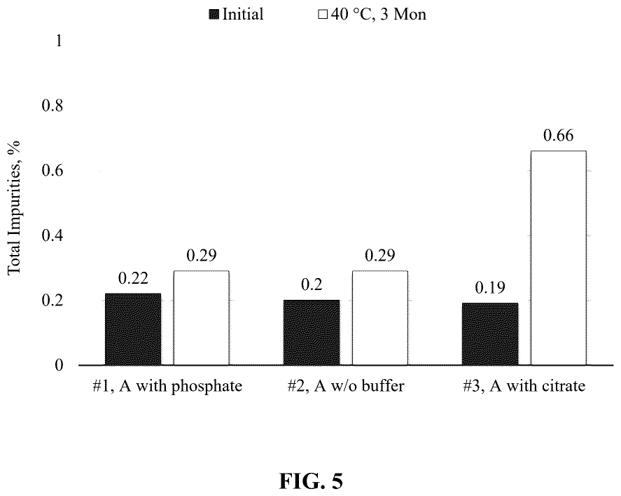

The stability of Formulation A (see Table 3-1 above) with 10 mg/mL midazolam was studied at 40 °C for 3 months for pH, impurity, and assay recovery. Formulation A was compared to a formulation without a buffer agent and a formulation with a citrate buffer. The results are shown in Table 4. FIG. 4 shows the pH stability of the midazolam solution, and FIG. 5 shows the total impurities of the midazolam solution at 40 °C, 3 months.

Assay recovery data in Table 4-1 indicates that midazolam solubility for all three lots did not change significantly over 3 months at 40 °C; additionally, all three solutions displayed comparable assay recovery percentages. The USP criteria for midazolam injection solutions for assay recovery is defined as being from 90.0% to 110.0%. Therefore, all three lots demonstrated excellent assay recovery percentages.

The pH change data of the solutions in Table 4-1 was measured and compared (see FIG. 4). The data indicates that the solutions with a buffer agent were more stable than the solution without a buffer over 3 months at 40 °C (FIG. 4).

The citrate buffer in the midazolam solution (Item 3) led to an unknown impurity at a relative retention time of 0.24 over 0.3% of the midazolam peak area that is not present in the other two solutions. Items 1 (the formulation with phosphate buffer) and 2 (the formulation without buffer) had a lower increase of total impurities from 0.22 to 0.29 and 0.20 to 0.29, respectively. Item 3 (the formulation with citrate buffer), however, displayed a large increase in impurities of nearly 247%, indicating an unstable stability profile for the citrate buffer system. The USP criteria for midazolam injection solutions for total impurities is defined as being no more than 1.0%. While all three lots had acceptable total impurity percentages, Item 1 had the most stable percentage of total impurities from the beginning to the end of the test.

The formulation using a phosphate buffer (Item 1) demonstrated greater stability and outperformed the formulation with a citrate buffer and the formulation without a buffer. The formulation with a phosphate buffer demonstrated a comparable assay recovery percentage to the other two formulations while having both a low change in pH and a low amount of impurities.

TABLE 4-1
Stability Results of Aqueous Intranasal Midazolam Formulations

| | | | | Impurity, as Percentage of the Midazolam Peak | | | | | | | |
|---|---|---|---|---|---|---|---|---|---|---|---|
| | Formulation | Storage | | Imp. | Imp. | Imp. | Imp. | Imp. | Imp. | Total | Assay |
| # | Info. | Condition^{a} | pH | #1^{b} | #2^{c} | #3^{d} | #4^{e} | #5^{f} | #6^{g} | Imp. | Recovery |
| 1 | A w/ | Initial | 3.63 | 0.00 | 0.04 | 0.04 | 0.04 | 0.04 | 0.06 | 0.22 | |
| | Phosphate | 3 months | 3.68 | 0.00 | 0.04 | 0.04 | 0.10 | 0.04 | 0.07 | 0.29 | 98.7% |
| | buffer | | | | | | | | | | |
| 2 | A w/o | Initial | 3.63 | 0.00 | 0.04 | 0.04 | 0.02 | 0.03 | 0.07 | 0.20 | |
| | buffer | 3 months | 3.73 | 0.00 | 0.04 | 0.04 | 0.10 | 0.04 | 0.07 | 0.29 | 96.4% |
| 3 | A w/ | Initial | 3.59 | 0.00 | 0.04 | 0.04 | 0.02 | 0.03 | 0.06 | 0.19 | |
| | Citrate | 3 months | 3.63 | 0.39 | 0.04 | 0.05 | 0.06 | 0.04 | 0.08 | 0.66 | 98.7% |
| | buffer | | | | | | | | | | |

^{a}Storage conditions: 40 °C, 75% Relative Humidity
^{b}Impurity #1: Unknown, relative retention time (RRT) at 0.24
^{c}Impurity #2: Midazolam Oxide, RRT at 0.55
^{d}Impurity #3: Midazolam Oxide, RRT at 0.57
^{e}Impurity #4: 5,6-Dihydro Midazolam, RRT at 0.82
^{f}Impurity #5: Enantiomer of 5,6-Dihydro Midazolam, RRT at 0.86
^{g}Impurity #6: Desfluoromidazolam, RRT at 1.22

### Example 5: Intranasal Midazolam Formulation Stability in Varying Temperatures

Formulation A of both 10 mg/mL midazolam and 20 mg/mL midazolam were further tested with different storage temperatures at either 4 °C, 25 °C, or 40 °C for 3 months.

The data in Table 5-1 displays the stability of Formulation A in all three temperatures over the 3 months. The average pH change for the three storage conditions was an increase of about 0.09 pH. The average change in the percentage of total impurities for the three storage conditions was an increase of about 0.05%. The assay recovery was also measured to be from 97.2% to 102.1%. The three noted criteria demonstrate that the formulation is chemically and physically stable at low, room, and high temperatures.

TABLE 5-1
Midazolam Formulation Stability in Varying Temperatures

| | | | Total | | |
|---|---|---|---|---|---|
| | | | Impurities, | Assay Recovery | |
| Lot No. | Storage Condition | pH | % | vs. Initial | Appearance |
| M 002_071124 | 0 time | 3.63 | 0.22 | | Clear Solution |
| 10 mg/mL | 4 °C, 3 months | 3.66 | 0.25 | 100.3% | Clear Solution |
| | 25 °C, 3 months | 3.69 | 0.25 | 97.2% | Clear Solution |
| | 40 °C, 3 months | 3.68 | 0.31 | 98.7% | Clear Solution |
| M 002_111324 | 0 time | 3.62 | 0.21 | | Clear Solution |
| 20 mg/mL | 4 °C, 3 months | 3.77 | 0.25 | 102.1% | Clear Solution |
| | 25 °C, 3 months | 3.78 | 0.26 | 101.8% | Clear Solution |
| | 40 °C, 3 months | 3.71 | 0.26 | 101.1% | Clear Solution |

### Example 6: Formulation Degradation Comparison Between Intranasal Midazolam Formulation with Phosphate Buffer and Intranasal Midazolam Formulations with Lactic Acid

A degradation study was conducted at 60 °C for 5 days on an intranasal midazolam formulation using a phosphate buffer system and two samples of the prior art intranasal midazolam formulation disclosed in U S 2021/0379080 A1 comprising midazolam at 5% w/w, polysorbate 80 at 2.5% w/w, disodium edetate at 0.1% w/w, benzalkonium chloride at 0.02% w/w, purified water, sodium hydroxide solution in an amount sufficient to make the solution pH 3.2, and lactic acid in 10.5% w/w as the buffer system. The test evaluated the proportion of impurities found in each formulation.

As demonstrated in Table 6-1 below, the formulations with lactic acid were prepared at a pH of about 3.2 and 3.5, respectively. At the relative retention time (RRT) of 0.42, the two formulations with a lactic buffer system had large increases in impurities from 0% to 0.65% and 0% to 0.39%, respectively. In contrast, the formulation with a phosphate buffer system had no increase in the same RRT of 0.42.

TABLE 6-1
Midazolam Solutions with Different Buffer Systems at 60 °C 5 days

| | | | | | Midazolam HPLC Impurities, % | | | |
|---|---|---|---|---|---|---|---|---|
| | | | | | at | at | at | at |
| Formulation | | Midazolam | Storage | | RRT | RRT | RRT | RRT |
| Information | API | Concentration | Condition | pH | 0.42 | 0.55 | 0.57 | 0.82 |
| Formulation A | Midazolam | 10 mg/mL | 0 time | 3.61 | 0.00 | 0.04 | 0.05 | 0.00 |
| with Phosphate | HCl | | 60 °C, 5 days | 3.69 | 0.00 | 0.04 | 0.04 | 0.09 |
| Buffer | | | | | | | | |
| US | Midazolam | 50 mg/mL | 0 time | 3.19 | 0.00 | 0.00 | 0.00 | 0.07 |
| 2021/0379080A 1, | | | 60 °C, 5 days | 3.26 | 0.65 | 0.00 | 0.00 | 0.09 |
| Formulation with | | | | | | | | |
| Lactic Acid | | | | | | | | |
| US | Midazolam | 50 mg/mL | 0 time | 3.49 | 0.00 | 0.00 | 0.00 | 0.06 |
| 2021/0379080A 1, | | | 60 °C, 5 days | 3.53 | 0.39 | 0.00 | 0.00 | 0.12 |
| Formulation with | | | | | | | | |
| Lactic Acid | | | | | | | | |

| | | | | | |
|---|---|---|---|---|---|
| USP Specification for Midazolam Injection | 3.0-4.0 | ≤0.1 | ≤0.1 | ≤0.1 | ≤0.5 |

| | Midazolam HPLC Impurities, % | | | |
|---|---|---|---|---|
| | at | at | at | Total |
| Formulation | RRT | RRT | RRT | Impurities |
| Information | 0.86 | 1.22 | 1.77 | (>0.05%), % |
| Formulation A | 0.04 | 0.07 | 0.00 | 0.20 |
| with Phosphate | 0.04 | 0.07 | 0.00 | 0.28 |
| Buffer | | | | |
| US | 0.00 | 0.09 | 0.00 | 0.16 |
| 2021/0379080A 1, | 0.00 | 0.10 | 0.17 | 1.01 |
| Formulation with | | | | |
| Lactic Acid | | | | |
| US | 0.00 | 0.08 | 0.00 | 0.14 |
| 2021/0379080A 1, | 0.00 | 0.11 | 0.24 | 0.86 |
| Formulation with | | | | |
| Lactic Acid | | | | |
| USP Specification for | ≤0.5 | ≤0.1 | ≤0.1 | ≤1.0 |
| Midazolam Injection | | | | |

The formulations with a lactic acid buffer system had a large increase in total impurities at the end of the study of 0.16% to 1.01% and 0.14% to 0.86%, respectively. In contrast, the formulation with a phosphate buffer system demonstrated a smaller increase in total impurities from 0.20% to 0.28%. Moreover, the 1.01% of the formulation with a lactic acid buffer system prepared at a pH of about 3.2 is slightly above the upper limit of the USP criteria for midazolam injection solutions for total impurities, which is 1.0%. The formulations with lactic acid also showed several unknown impurities that were out of specification.

### CITATIONS TO REFERENCES

Below are the complete citations for references listed above:
Behl, C. R., et al. “Effects of physicochemical properties and other factors on systemic nasal drug delivery,” Advanced Drug Delivery Reviews 29 (1998) 89-116.
Deot N, et al. “Effect of Intranasal Corticosteroids on Secondary Sinonasal Symptoms: A Systematic Review of Randomized Trials.” Amj Rhinol Allergy. 2019 September; 33(5):601-607. doi: 10.1177/1945892419844397. Epub 2019 Apr. 30. PMID: 31035786.
Gudmundsdottir H, et al., “Intranasal administration of midazolam in a cyclodextrin based formulation: bioavailability and clinical evaluation in humans.” Pharmazie. 2001 December; 56(12):963-6. PMID: 11802661.
“Hydrogen Chloride.” Centers for Disease Control and Prevention, Centers for Disease Control and Prevention, 210 ct. 2014, wwwn.cdc.gov/TSP/MMG/MMGDetails.aspx?mmgid=758&toxid=147 #.-:text=Hydrogen %20 chloride %20gas%20is%20intensely,of%20the%20throat%20and%20suffocation.
Knoester, P D, et al. “Pharmacokinetics and Pharmacodynamics of Midazolam Administered as a Concentrated Intranasal Spray. A Study in Healthy Volunteers.” Br J Clin Pharmacol. 2002 May; 53(5):501-507.
“Lactic Acid: Uses, Tips, Faqs & M ore.” GoodRx, www.goodrx.com/lactic-acid/what-is. Accessed 4 Nov. 2024.
Lingamchetty T N, Hosseini S A, Saadabadi A. “Midazolam.” [Updated 2023 J un 5]. In: StatPearls [Internet]. Treasure Island (FL): StatPearls Publishing; 2024 January-. A vailable from: https://www.ncbi.nlm.nih.gov/books/NBK537321/
Lui et. al, “Intranasal Absorption of Flurazepam, Midazolam, and Triazolam in Dogs”, J. of Pharmaceutical Sciences, 1991, 80(12):1125-9.
Merkus et al. “Classification of Cilio-Inhibiting Effects of Nasal Drugs”, Laryngoscope 111:595-602, 2001.
Pires, P. C., et al. “Strategies to Improve Drug Strength in Nasal Preparations for Brain Delivery of Low Aqueous Solubility Drugs.” M DPI, M ultidisciplinary Digital Publishing Institute, 8 Mar. 2022, www.mdpi.com/1999-4923/14/3/588.
Prommer E. “Midazolam: an essential palliative care drug.” Palliat Care Soc Pract. 2020 J an 13; 14:2632352419895527. doi: 10.1177/2632352419895527. PMID: 32215374; PMCID: PMC7065504.
Thorat, Santosh. “Formulation and Product Development of N asal Spray: A n Overview.” Scholars Journal of Applied Medical Sciences, August 2016; 4(8D):2976-2985
Wermeling, D P., et al. “Pharmacokinetics and Pharmacodynamics of a New Intranasal Midazolam Formulation in Healthy Volunteers”. Anesthesia & Analgesia 103(2):p 344-349, August 2006.|DOI: 10.1213/01.ane.0000226150.90317.16

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

### Embodiments

The invention provides also the following non-limiting embodiments.

Embodiment 1 is an aqueous pharmaceutical formulation of midazolam solution for intranasal administration comprising: a. midazolam, or a pharmaceutically acceptable salt form; b. not less than 75% (w/w) of water relative to the total weight of the formulation; c. a co-solvent or a combination of co-solvents selected from the group consisting of ethanol, isopropyl alcohol, propylene glycol, ethylene glycol, glycerol, povidone, 2-(2-ethoxyethoxy)ethanol, or combinations thereof; d. a surfactant selected from the group consisting of polyethylene glycols, cyclodextrins, polysorbates, and sorbitan oleates; e. a preservative selected from the group consisting of phenol, benzyl alcohol, benzalkonium chloride, and vitamin E; f. a stabilizing agent selected from the group consisting of ethylenediaminetetraacetic acid (EDTA), ethylglycoltetraacetic acid (EGTA), citric acid or combinations thereof; g. an isotonic agent selected from the list consisting of sodium chloride, potassium chloride, calcium chloride, or mixtures thereof; and h. pH buffer agents selected from the group consisting of citrate buffer, acetate buffer, phosphate buffer, or mixtures thereof in an amount sufficient to adjust the solution pH between about 3.2 and about 4.2.

Embodiment 2 is the pharmaceutical formulation of midazolam solution of embodiment 1, wherein the midazolam, or a pharmaceutically acceptable salt form thereof, is midazolam hydrochloride.

Embodiment 3 is the aqueous pharmaceutical formulation of midazolam solution of embodiment 2, wherein said midazolam hydrochloride is present in a concentration of about 5 mg/mL to about 40 mg/mL.

Embodiment 4 is the aqueous pharmaceutical formulation of midazolam solution of any one of embodiments 1 to 3, wherein the co-solvent is a combination of ethanol and propylene glycol.

Embodiment 5 the aqueous pharmaceutical formulation of midazolam solution of embodiment 4, wherein the combination of ethanol and propylene glycol is present in an amount of about 0.1% to about 20% (w/w) relative to the total weight of the formulation.

Embodiment 6 is the aqueous pharmaceutical formulation of midazolam solution of any one of embodiments 1 to 5, wherein the surfactant is polysorbate 20 (polyoxyethylene (20) sorbitan monolaurate).

Embodiment 7 is the aqueous pharmaceutical formulation of midazolam solution of embodiment 6, wherein the polysorbate 20 (polyoxyethylene (20) sorbitan monolaurate) is present in an amount of about 0.1% to about 20% (w/w) relative to the total weight of the formulation.

Embodiment 8 is the aqueous pharmaceutical formulation of midazolam solution of any one of embodiments 1 to 7, wherein the preservative is benzalkonium chloride.

Embodiment 9 is the aqueous pharmaceutical formulation of midazolam solution of embodiment 8, wherein the benzalkonium chloride is present in an amount not more than about 0.01% (w/w) relative to the total weight of the formulation.

Embodiment 10 is the aqueous pharmaceutical formulation of midazolam solution of any one of embodiments 1 to 9, wherein the stabilizing agent is ethylenediaminetetraacetic acid (EDTA).

Embodiment 11 is the aqueous pharmaceutical formulation of midazolam solution of embodiment 10, wherein the EDTA is present in an amount not more than about 0.02% (w/w) relative to the total weight of the formulation.

Embodiment 12 is the aqueous pharmaceutical formulation of midazolam solution of any one of embodiments 1 to 11, wherein the isotonic agent is sodium chloride.

Embodiment 13 is the aqueous pharmaceutical formulation of midazolam solution of embodiment 12, wherein the sodium chloride is present in an amount not more than about 0.9% (w/w) relative to the total weight of the formulation.

Embodiment 14 is the aqueous pharmaceutical formulation of midazolam solution of any one of embodiments 1 to 13, wherein the mixture of pH buffer agents comprises sodium phosphate monobasic and phosphoric acid.

Embodiment 15 is the aqueous pharmaceutical formulation of midazolam solution of embodiment 14, wherein the sodium phosphate monobasic is present in an amount of about 0.01% to about 1.0% (w/w) relative to the total weight of the formulation and phosphoric acid is present in an amount sufficient to adjust the formulation pH to be between about 3.2 and about 4.2.

Embodiment 16 is the aqueous pharmaceutical formulation of midazolam solution of any one of embodiments 1 to 15, wherein the density of the solution is similar to that of water of 1 g/mL and not more than 1.05 g/mL.

Embodiment 17 is the aqueous pharmaceutical formulation of midazolam solution of any one of embodiments 1 to 16, wherein the viscosity of the solution is between about 1 cP to about 10 cP.

Embodiment 18 is the aqueous pharmaceutical formulation of midazolam solution of any one of embodiments 1 to 17, wherein the osmolality of the solution is between about 290 mOsm/kg to about 400 mOsm/kg.

Embodiment 19 is the aqueous pharmaceutical formulation of midazolam solution of any one of embodiments 1 to 18, is delivered by an intranasal device at the dose of about 0.1 mg to about 5 mg of midazolam.

Embodiment 20 is the aqueous pharmaceutical formulation of midazolam solution of any one of embodiments 1 to 19, is delivered by an intranasal device at the volume of about 0.1 mL to about 0.7 mL.

Embodiment 21 is an aqueous pharmaceutical formulation of midazolam solution for intranasal administration comprising: a. midazolam hydrochloride in a solution at the concentration of midazolam between about 10 mg/mL to about 20 mg/mL; b. water in an amount ranging from about 75% to about 95% (w/w) relative to the total weight of the formulation; c. ethanol in an amount ranging from about 9% to about 10% (w/w) relative to the total weight of the formulation; d. propylene glycol in an amount ranging from about 7% to about 8% (w/w) relative to the total weight of the formulation; e. polysorbate 20 (polyoxyethylene (20) sorbitan monolaurate) in an amount not more than about 2.5% (w/w) relative to the total weight of the formulation; f. benzalkonium chloride in an amount ranging from about 0.001% to about 0.01% (w/w) relative to the total weight of the formulation; g. EDTA in an amount ranging from about 0.001% to about 0.02% (w/w) relative to the total weight of the formulation; h. sodium chloride in an amount ranging from about 0.1% to about 0.9% (w/w) relative to the total weight of the formulation; and i. sodium phosphate monobasic and phosphoric acid wherein sodium phosphate monobasic in an amount ranging from about 0.1% to about 1.0% (w/w) relative to the total weight of the formulation, and wherein phosphoric acid in an amount sufficient to adjust the solution pH to be between about 3.2 and about 4.2.

Embodiment 22 is an aqueous pharmaceutical formulation of midazolam solution for intranasal administration comprising: a. midazolam hydrochloride in a solution at the concentration of midazolam of about 10 mg/mL; b. water in an amount of about 78.8% (w/w) relative to the total weight of the formulation; c. ethanol in an amount of about 9.5% (w/w) relative to the total weight of the formulation; d. propylene glycol in an amount of about 7.3% (w/w) relative to the total weight of the formulation; e. polysorbate 20 (polyoxyethylene (20) sorbitan monolaurate) in an amount of about 2.39% (w/w) relative to the total weight of the formulation; f. benzalkonium chloride in an amount of about 0.01% (w/w) relative to the total weight of the formulation; g. EDTA in an amount of about 0.02% (w/w) relative to the total weight of the formulation; h. sodium chloride in an amount of about 0.51% (w/w) relative to the total weight of the formulation; and i. sodium phosphate monobasic and phosphoric acid wherein sodium phosphate monobasic in an amount of about 0.36% (w/w) relative to the total weight of the formulation and phosphoric acid in an amount sufficient to adjust the solution pH to be between about 3.5 and about 3.8.

Embodiment 23 is an aqueous pharmaceutical formulation of midazolam solution for intranasal administration comprising: a. midazolam hydrochloride in a solution at the concentration of midazolam of about 20 mg/mL; b. water in an amount of about 77.7% (w/w) relative to the total weight of the formulation; c. ethanol in an amount of about 9.5% (w/w) relative to the total weight of the formulation; d. propylene glycol in an amount of about 7.3% (w/w) relative to the total weight of the formulation; e. polysorbate 20 (polyoxyethylene (20) sorbitan monolaurate) in an amount of about 2.39% (w/w) relative to the total weight of the formulation; f. benzalkonium chloride in an amount of about 0.01% (w/w) relative to the total weight of the formulation; g. EDTA in an amount of about 0.02% (w/w) relative to the total weight of the formulation; h. sodium chloride in an amount of about 0.51% (w/w) relative to the total weight of the formulation; and i. sodium phosphate monobasic and phosphoric acid where sodium phosphate monobasic in an amount of about 0.36% (w/w) relative to the total weight of the formulation and phosphoric acid in an amount sufficient to adjust the solution pH to be between about 3.5 and about 3.8.

Embodiment 24 is the aqueous pharmaceutical formulation of midazolam solution of any one of embodiments 21 to 23, wherein the formulation is stable at 4C, 25 °C, and 40 °C with total impurities not more than 0.30% after 3 months of storage.

1. An aqueous pharmaceutical formulation of midazolam solution for intranasal administration comprising:
a. midazolam, or a pharmaceutically acceptable salt form at a concentration of midazolam between about 10 mg/mL to about 20 mg/mL;
b. water in an amount ranging from about 7500 to about 95% (w/w) relative to the total weight of the formulation;
c. from about 5% to about 20% w/w of a co-solvent or a combination of co-solvents selected from the group consisting of ethanol, isopropyl alcohol, propylene glycol, ethylene glycol, glycerol, povidone, 2-(2-ethoxyethoxy)ethanol, or combinations thereof,
d. a surfactant selected from the group consisting of polyethylene glycols, cyclodextrins, polysorbates, and sorbitan oleates;
e. a preservative selected from the group consisting of phenol, benzyl alcohol, benzalkonium chloride, and vitamin E;
f. a stabilizing agent selected from the group consisting of ethylenediaminetetraacetic acid (EDTA), ethylglycoltetraacetic acid (EGTA), citric acid or combinations thereof,
g. an isotonic agent selected from the list consisting of sodium chloride, potassium chloride, calcium chloride, or mixtures thereof; and
h. pH buffer agents selected from the group consisting of citrate buffer, acetate buffer, phosphate buffer, or mixtures thereof in an amount sufficient to adjust the solution pH between about 3.2 and about 4.2.

2. The pharmaceutical formulation of midazolam solution of claim 1, wherein the midazolam, or a pharmaceutically acceptable salt form thereof, is midazolam hydrochloride.

3. The aqueous pharmaceutical formulation of midazolam solution of claim 2, wherein said midazolam hydrochloride is present in a concentration of about 10 mg/mL or about 20 mg/mL.

4. The aqueous pharmaceutical formulation of midazolam solution of claim 1, wherein the co-solvent is a combination of the co-solvents.

5. The aqueous pharmaceutical formulation of midazolam solution of claim 4, wherein the combination of co-solvent is a combination of ethanol and propylene glycol.

6. The aqueous pharmaceutical formulation of midazolam solution of claim 1, wherein the surfactant is polysorbate 20 (polyoxyethylene (20) sorbitan monolaurate).

7. The aqueous pharmaceutical formulation of midazolam solution of claim 6, wherein the polysorbate 20 (polyoxyethylene (20) sorbitan monolaurate) is present in an amount of about 0.1% to about 20% (w/w) relative to the total weight of the formulation.

8. The aqueous pharmaceutical formulation of midazolam solution of claim 1, wherein the preservative is benzalkonium chloride.

9. The aqueous pharmaceutical formulation of midazolam solution of claim 8, wherein the benzalkonium chloride is present in an amount not more than about 0.01% (w/w) relative to the total weight of the formulation.

10. The aqueous pharmaceutical formulation of midazolam solution of claim 1, wherein the stabilizing agent is ethylenediaminetetraacetic acid (EDTA).

11. The aqueous pharmaceutical formulation of midazolam solution of claim 10, wherein the EDTA is present in an amount not more than about 0.02% (w/w) relative to the total weight of the formulation.

12. The aqueous pharmaceutical formulation of midazolam solution of claim 1, wherein the isotonic agent is sodium chloride.

13. The aqueous pharmaceutical formulation of midazolam solution of claim 12, wherein the sodium chloride is present in an amount not more than about 0.9% (w/w) relative to the total weight of the formulation.

14. The aqueous pharmaceutical formulation of midazolam solution of claim 1, wherein the mixture of pH buffer agents comprises sodium phosphate monobasic and phosphoric acid.

15. The aqueous pharmaceutical formulation of midazolam solution of claim 14, wherein the sodium phosphate monobasic is present in an amount of about 0.01% to about 1.0% (w/w) relative to the total weight of the formulation and phosphoric acid is present in an amount sufficient to adjust the formulation pH to be between about 3.2 and about 4.2.

16. The aqueous pharmaceutical formulation of midazolam solution of claim 1, wherein the density of the solution is similar to that of water of 1 g/mL and not more than 1.05 g/mL.

17. The aqueous pharmaceutical formulation of midazolam solution of claim 1, wherein the viscosity of the solution is between about 1 cP to about 10 cP.

18. The aqueous pharmaceutical formulation of midazolam solution of claim 1, wherein the osmolality of the solution is between about 290 mOsm/kg to about 400 mOsm/kg.

19. The aqueous pharmaceutical formulation of midazolam solution of claim 1, is delivered by an intranasal device at the dose of about 0.1 mg to about 5 mg of midazolam.

20. The aqueous pharmaceutical formulation of midazolam solution of claim 1, is delivered by an intranasal device at the volume of about 0.1 mL to about 0.7 mL.

21. An aqueous pharmaceutical formulation of midazolam solution for intranasal administration comprising:
a. midazolam hydrochloride in a solution at the concentration of midazolam between about 10 mg/mL to about 20 mg/mL;
b. water in an amount ranging from about 75% to about 95% (w/w) relative to the total weight of the formulation;
c. ethanol in an amount ranging from about 9% to about 10% (w/w) relative to the total weight of the formulation;
d. propylene glycol in an amount ranging from about 7% to about 8% (w/w) relative to the total weight of the formulation;
e. polysorbate 20 (polyoxyethylene (20) sorbitan monolaurate) in an amount not more than about 2.5% (w/w) relative to the total weight of the formulation;
f. benzalkonium chloride in an amount ranging from about 0.001% to about 0.01% (w/w) relative to the total weight of the formulation;
g. EDTA in an amount ranging from about 0.001% to about 0.02% (w/w) relative to the total weight of the formulation;
h. sodium chloride in an amount ranging from about 0.1% to about 0.9% (w/w) relative to the total weight of the formulation; and
i. sodium phosphate monobasic and phosphoric acid wherein sodium phosphate monobasic is in an amount ranging from about 0.1% to about 1.0% (w/w) relative to the total weight of the formulation, and wherein phosphoric acid in an amount sufficient to adjust the solution pH to be between about 3.2 and about 4.2.

22. An aqueous pharmaceutical formulation of midazolam solution for intranasal administration comprising:
a. midazolam hydrochloride in a solution at the concentration of midazolam of about 10 mg/mL;
b. water in an amount of about 78.8% (w/w) relative to the total weight of the formulation;
c. ethanol in an amount of about 9.5% (w/w) relative to the total weight of the formulation;
d. propylene glycol in an amount of about 7.3% (w/w) relative to the total weight of the formulation;
e. polysorbate 20 (polyoxyethylene (20) sorbitan monolaurate) in an amount of about 2.39% (w/w) relative to the total weight of the formulation;
f. benzalkonium chloride in an amount of about 0.01% (w/w) relative to the total weight of the formulation;
g. EDTA in an amount of about 0.02% (w/w) relative to the total weight of the formulation;
h. sodium chloride in an amount of about 0.51% (w/w) relative to the total weight of the formulation; and
i. sodium phosphate monobasic and phosphoric acid wherein sodium phosphate monobasic in an amount of about 0.36% (w/w) relative to the total weight of the formulation and phosphoric acid in an amount sufficient to adjust the solution pH to be between about 3.5 and about 3.8.

23. An aqueous pharmaceutical formulation of midazolam solution for intranasal administration comprising:
a. midazolam hydrochloride in a solution at the concentration of midazolam of about 20 mg/mL;
b. water in an amount of about 77.7% (w/w) relative to the total weight of the formulation;
c. ethanol in an amount of about 9.5% (w/w) relative to the total weight of the formulation;
d. propylene glycol in an amount of about 7.3% (w/w) relative to the total weight of the formulation;
e. polysorbate 20 (polyoxyethylene (20) sorbitan monolaurate) in an amount of about 2.39% (w/w) relative to the total weight of the formulation;
f. benzalkonium chloride in an amount of about 0.01% (w/w) relative to the total weight of the formulation;
g. EDTA in an amount of about 0.02% (w/w) relative to the total weight of the formulation;
h. sodium chloride in an amount of about 0.51% (w/w) relative to the total weight of the formulation; and
i. sodium phosphate monobasic and phosphoric acid where sodium phosphate monobasic in an amount of about 0.36% (w/w) relative to the total weight of the formulation and phosphoric acid in an amount sufficient to adjust the solution pH to be between about 3.5 and about 3.8.

24. The aqueous pharmaceutical formulation of midazolam solution of claim 21, wherein the formulation is stable at 4 °C, 25 °C, and 40 °C with total impurities not more than 0.30% after 3 months of storage.
